(19) United States
(12) Patent Application Publication
(10) Pub. No.: US 2025/0352116 A1
(43) Pub. Date: Nov. 20, 2025

(54) METHOD AND SYSTEM FOR CONFIGURING ELECTRODES FOR EVOKED NEURAL RESPONSE MEASUREMENT

(71) Applicant: Saluda Medical Pty Limited, Macquarie Park, New South Wales (AU)

(72) Inventor: Gerrit Gmel, Macquarie Park (AU)

(21) Appl. No.: 18/871,340

(22) PCT Filed: Jun. 7, 2023
(86) PCT No.: PCT/AU2023/050501

§ 371 Date: Dec. 3, 2024

(30) Foreign Application Priority Data
Jun. 7, 2022 (AU) 2022901556

(51) Int. Cl. A61B 5/294; A61B 5/00; A61B 5/311; A61B 5/388; A61N 1/36

(52) U.S. Cl. CPC A61B 5/294; A61B 5/311; A61B 5/388; A61B 5/6877; A61B 5/7203; A61N 1/36053; A61N 1/36071; A61N 1/3615

(57) ABSTRACT

Disclosed is a method and system for selecting a reference electrode wherein at least one electrode of an electrode assembly is configured in a sensing mode and is connected to a sensing circuitry configured to measure a neural potential. Further, the electrodes configured in the sensing mode includes at least one measuring electrode and at least one reference electrode. The method and system can configure the electrodes of the electrode assembly based on conditions related to the stimulation and neural measurement.

The present application claims priority from Australian Provisional Patent Application No 2022901556 filed on 7 Jun. 2022, the contents of which are incorporated herein by reference in their entirety.

### TECHNICAL FIELD

The present invention relates to the area of implantable electrical stimulation systems, particularly to electrode assemblies configured to deliver stimulation to a portion of the tissue and/or a target nerve.

### BACKGROUND OF THE INVENTION

There is a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes or elicits a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, is therefore necessary to apply stimuli with intensity below a comfort threshold, above which uncomfortable or painful percepts arise due to over-recruitment of Aβ fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the comfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation systems of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be generated by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO 2012/155183, the content of which is incorporated herein by reference.

Applying neural stimulation therapy to pelvic floor disorders is well known in the industry. Pelvic floor disorders include functions that are influenced by sacral nerves. For example, functions such as: urinary incontinence, urinary urge/frequency, urinary retention, pelvic pain, bowel dysfunction (constipation, diarrhea), and sexual dysfunction are some of the functions influenced by the sacral nerves.

There are many differences between spinal cord stimulation (SCS) and sacral nerve stimulation (SNS). Firstly, the anatomy is different in the spinal cord and the pelvic floor. Secondly, the dimension and the physical properties of leads change drastically between the two modalities. Further, one other significant difference between the SNS application and the SCS application is that the target nerve in SNS is a mixed nerve while the neural pathway in SCS is predominantly composed of Aβ fibres. Therefore, rather than having one fibre type (A-beta fibres), in SNS, a multitude of fibre types will be activated by stimulation. This is explained further in WO2019204884, the content of which is incorporated in its entirety by reference in this application.

It is, therefore, important to determine the type of fibres recruited due to the stimulation of the pelvic floor in order to provide the best outcome for the patient. Further, stimulating other fibres in a mixed nerve could result in unpleasant side effects in the patient.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein is a method of selecting a reference electrode in the electrode array based on the location of an electrode relative to a target nerve. Electrodes in the electrode array can be configured in a stimulation mode and/or in a sensing mode. the electrodes configured in the stimulation mode apply an electrical stimulus to a portion of tissue. Further, the electrodes configured in the sensing mode are configured to measure a neural potential. More specifically, disclosed is a method of selecting a reference electrode as an electrode, in the electrode array, that is located at a maximum distance from a target nerve and/or senses the least amount of neural potential elicited in the target nerve by the application of stimulation, as a reference electrode. The reference electrode is chosen such that it results in a monopolar recording of an evoked neural potential. The reference electrode is chosen to be an electrode that is ‘indifferent’ to (unaffected by) both the transients produced during the application of the stimulus and the evoked neural response.

According to a first aspect of the present technology, there is provided a neural stimulation system comprising: an implantable neuromodulation device for controllably delivering neural stimuli: an electrode assembly electronically coupled to the implantable neuromodulation device and having a set of electrodes proximal to a distal end of the electrode assembly, the implantable neuromodulation device comprising: stimulation circuitry for applying the neural stimuli to at least one target nerve, wherein the stimuli elicit a neural potential from a target nerve: measurement circuitry configured to process signals sensed subsequent to respective neural stimuli at a pair of sensing electrodes of the set of electrodes; and a control unit configured to control the stimulation circuitry to apply the neural stimuli; and a processor configured to: configure a plurality of electrodes of the set of electrodes in at least one of a simulation mode and a sensing mode, wherein the electrodes configured in the stimulation mode are connected to the stimulation circuitry and the electrodes configured in the sensing mode are connected to the measurement circuitry, and wherein the plurality of electrodes configured in the sensing mode comprise a reference electrode; and select an electrode from the set of electrodes as the reference electrode based on a distance of the electrode from the target nerve.

The processor may be configured to select the electrode of the set of electrodes that is located at a maximum distance from the target nerve as the reference electrode.

The plurality of electrodes configured in the stimulation mode may comprise a stimulation electrode, wherein the processor is further configured to select the reference electrode based on the distance between the reference electrode and the stimulation electrode.

The processor may be further configured to determine the distance between the stimulation electrode and the reference electrode using impedance measurements.

The target nerve may include nerve fibres such as sacral nerve, vagus nerve, and nerve fibres in a dorsal column.

The processor may be further configured to process one or more characteristics of the neural potential to determine the distance of the electrode from the target nerve. The one or more characteristics of the neural potential may include a conduction velocity, latency, dilation, peak-to-peak ratio and an amplitude.

The neural stimulation system may further comprise a remote device in communication with the implantable neuromodulation device. The processor may be part of the remote device.

The processor forms part of the implantable neuromodulation device.

In a second aspect of the present technology, there is provided a remote device in communication with an implantable neuromodulation device, the remote device comprising: a processing unit configured to receive instructions from a user: a communication unit configured to send and receive instructions to and from the implantable neuromodulation device, the processing unit configured to send instructions to the implantable neuromodulation device to: configure a plurality of electrodes of a set of electrodes in at least one of a stimulation mode and a sensing mode, wherein the electrodes configured in the sensing mode comprise a reference electrode; and select an electrode from the set of electrodes as the reference electrode based on a distance of the electrode from a target nerve.

The remote device may be one of a remote control, a portable computing device, and an external device. The target nerve may include nerve fibres such as sacral nerve, vagus nerve, and nerve fibres in a dorsal column.

In a third aspect of the present technology, there is provided a method of selecting a reference electrode, the method comprising: providing stimulation circuitry and measurement circuitry: providing a processing unit configured to control the stimulation circuitry and the measurement circuitry: providing a lead body having a proximal end and a distal end, the lead body having a set of electrodes proximal to the distal end: wherein the set of electrodes are configurable in at least one of a stimulation mode and a sensing mode: wherein the electrodes configured in the stimulation mode are connected to the stimulation circuitry, wherein the electrodes configured in the stimulation mode apply an electrical stimulus to a target nerve, wherein the electrical stimulus elicits a neural potential; and wherein the electrodes configured in the sensing mode are connected to the measurement circuitry, wherein the electrodes configured in the sensing mode include at least one reference electrode, wherein the electrodes configured in the sensing mode are configured to measure the elicited neural potential; and selecting an electrode from the set of electrodes as the reference electrode based on a distance of the electrode from the target nerve.

Selecting the electrode from the set of electrodes may comprise selecting the electrode that is located at a maximum distance from the target nerve as the reference electrode.

The electrodes configured in the stimulation mode may comprise a stimulation electrode, further comprising selecting the reference electrode based on the distance between the reference electrode and the stimulation electrode.

The method may further comprise determining the distance between the stimulation electrode and the reference electrode using impedance measurements.

The target nerve may include nerve fibres such as sacral nerve, vagus nerve, and nerve fibres in a dorsal column.

The method may further comprise processing one or more characteristics of the neural potential to determine the distance of the electrode from the target nerve.

One or more characteristics of the neural potential may include a conduction velocity, latency, dilation, peak-to-peak ratio and an amplitude.

In a fourth aspect of the present technology, there is provided a neural stimulation system comprising: an implantable neuromodulation device for controllably delivering neural stimuli, an electrode assembly electrically coupled to the implantable neuromodulation device, the electrode assembly including a set of electrodes proximal to a distal end of the electrode assembly, the implantable neuromodulation device comprising: stimulation circuitry for applying the neural stimuli to at least one target nerve, wherein the neural stimuli elicit a neural potential from a target nerve: measurement circuitry configured to process signals sensed subsequent to respective neural stimuli at a pair of sensing electrodes of the set of electrodes; and a control unit configured to control the stimulation circuitry to apply the neural stimuli; and a processor configured to: configure a plurality of electrodes of the set of electrodes in at least one of a simulation mode and a sensing mode, wherein the electrodes configured in the stimulation mode are connected to the stimulation circuitry and the electrodes configured in the sensing mode are connected to the measurement circuitry, wherein the plurality of electrodes configured in the sensing mode comprise a reference electrode; and select an electrode from the set of electrodes as the reference electrode such that the reference electrode senses an insubstantial amount of the elicited neural potential.

The plurality of electrodes configured in the sensing mode may comprise a recording electrode, and wherein the reference electrode senses an insubstantial amount of the elicited neural potential if the reference electrode senses less than 5% of the magnitude of the elicited neural potential sensed at the recording electrode.

The plurality of electrodes configured in the stimulation mode may comprise a return electrode, and wherein the processor is further configured to select the return electrode based on a desired level of a field at the target nerve.

The target nerve may include nerve fibres such as sacral nerve, vagus nerve, and nerve fibres in a dorsal column.

The neural stimulation system may further comprise a remote device in communication with the implantable neuromodulation device. The processor may be part of the remote device.

The processor may form part of the implantable neuromodulation device.

In a fifth aspect of the present technology, there is provided a remote device in communication with an implantable neuromodulation device, the remote device comprising: a processing unit configured to receive instructions from a user: a communication unit configured to send and receive instructions to and from the implantable neuromodulation device, the processing unit configured to send instructions to the implantable neuromodulation device to: configure a plurality of electrodes of a set of electrodes in at least one of a stimulation mode and a sensing mode, wherein the electrodes configured in the sensing mode comprise a reference electrode; and select an electrode from the set of electrodes as the reference electrode to sense an insubstantial amount of the elicited neural potential.

The plurality of electrodes configured in the sensing mode may comprise a recording electrode, and wherein the reference electrode senses an insubstantial amount of the elicited neural potential if the reference electrode senses less than 5% of the magnitude of the elicited neural potential sensed at the recording electrode.

The electrodes configured in the stimulation mode may comprise a return electrode, and wherein the processing unit is further configured to select the return electrode based on a desired level of a field at a target nerve. The target nerve includes nerve fibres such as sacral nerve, vagus nerve, and nerve fibres in a dorsal column.

The remote device may be one of a remote control, a portable computing device, and an external device.

In a sixth aspect of the present technology, there is provided a method of selecting a reference electrode, the method comprising: providing stimulation circuitry and measurement circuitry: providing a processing unit configured to control the stimulation circuitry and the measurement circuitry: providing for a lead body having a proximal end and a distal end, the lead body having a set of electrodes proximal to the distal end: wherein the set of electrodes are configurable in at least one of a stimulation mode and a sensing mode: wherein the electrodes configured in the stimulation mode are connected to the stimulation circuitry, wherein the electrodes configured in the stimulation mode apply an electrical stimulus to a target nerve, wherein the electrical stimulus elicits a neural potential; and wherein the electrodes configured in the sensing mode are connected to the measurement circuitry configured to measure the elicited neural potential, wherein the electrodes configured in the sensing mode include at least one reference electrode; and selecting an electrode from the set of electrodes as the reference electrode such that the reference electrode senses an insubstantial amount of the elicited neural potential.

The electrodes configured in the sensing mode may comprise a recording electrode, and wherein the reference electrode senses an insubstantial amount of the elicited neural potential if the reference electrode senses less than 5% of the magnitude of the elicited neural potential sensed at the recording electrode.

The electrodes configured in the stimulation mode may include a return electrode, further comprising selecting the return electrode based on a desired level of a field at the target nerve.

The target nerve may include nerve fibres such as sacral nerve, vagus nerve, and nerve fibres in a dorsal column.

In a seventh aspect of the present technology, there is provided a neural stimulation system comprising: an implantable neuromodulation device for controllably delivering neural stimuli, an electrode assembly electronically coupled to the implantable neuromodulation device, the electrode assembly having a proximal end and a distal end, the electrode assembly having a first set of electrodes at the distal end, a plurality of anchoring elements proximal to the first set of electrodes, and a second set of electrodes proximal to the anchoring elements, the implantable neuromodulation device comprising: stimulation circuitry for applying the neural stimuli to at least one target nerve, wherein the neural stimuli elicit a neural potential from a target nerve: measurement circuitry configured to process signals sensed subsequent to respective neural stimuli at a pair of sensing electrodes of the set of electrodes; and a control unit configured to control the stimulation circuitry to apply the neural stimuli; and a processor configured to: configure a plurality of electrodes of the first set of electrodes and the second set of electrodes in at least one of a simulation mode and a sensing mode, wherein the electrodes configured in the stimulation mode are connected to the stimulation circuitry and the electrodes configured in the sensing mode are connected to the measurement circuitry, and wherein the plurality of electrodes configured in the sensing mode comprise a reference electrode; and select an electrode from the first set or the second set of electrodes as the reference electrode based on a distance of the electrode from the target nerve.

The processor may be further configured to select the reference electrode from the second set of electrodes.

The plurality of electrodes configured in the sensing mode may comprise a recording electrode, and wherein the processor is further configured to select the recording electrode from the first set of electrodes.

The plurality of electrodes configured in the stimulation mode may comprise a return electrode, and wherein the processor is further configured to select the return electrode from the second set of electrodes.

In an eighth aspect of the present technology, there is provide a neural stimulation lead for applying stimulation to a tissue, the neural stimulation lead comprising: a lead body having a proximal end and a distal end: the lead body having a first set of electrodes at the distal end, a plurality of anchoring elements proximal to first set of electrodes and a second set of electrodes proximal to the anchoring elements: wherein the first set of electrodes and the second set of electrodes are configurable in at least one of a stimulation mode and a sensing mode: wherein the electrodes configured in the stimulation mode are connected to stimulation circuitry, wherein the electrodes configured in the stimulation mode apply an electrical stimulus to a target nerve, wherein the electrical stimulus elicits a neural potential: wherein the electrodes configured in the sensing mode are connected to measurement circuitry configured to measure the elicited neural potential, wherein the electrodes configured in the sensing mode include at least one reference electrode, wherein an electrode from the second set of electrodes is configured as the reference electrode.

The electrodes configured in the sensing mode may comprise a recording electrode, and wherein an electrode from the first set of electrodes is configured as the recording electrode.

The electrodes configured in the stimulation mode may include a return electrode, and wherein an electrode from the second set of electrodes is configured as the return electrode.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application-specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations will now be described with reference to the accompanying drawings, in which:

FIG. 1 schematically illustrates an implanted spinal cord stimulator, according to one implementation of the present technology;

FIG. 2 is a block diagram of the stimulator of FIG. 1:

FIG. 3 is a schematic illustrating interaction of the implanted stimulator of FIG. 1 with a nerve:

FIG. 4a illustrates an idealised activation plot for one posture of a patient undergoing neural stimulation;

FIG. 4b illustrates the variation in the activation plots with changing posture of the patient:

FIG. 5 is a schematic illustrating elements and inputs of a closed-loop neural stimulation (CLNS) system, according to one implementation of the present technology:

FIG. 6 illustrates the typical form of an electrically evoked compound action potential (ECAP) of a healthy subject:

FIG. 7 is a block diagram of a neural stimulation therapy system including the implanted stimulator of FIG. 1 according to one implementation of the present technology:

FIG. 8 is a block diagram illustrating the data flow of a neuromodulation therapy system such as the system of FIG. 7:

FIGS. 9A, 9B are side views of an electrode assembly for stimulating the pelvic floor nerves;

FIG. 9C is a perspective view of the electrode assembly of FIG. 9B;

FIG. 10 illustrates the distance between the nerve and the electrodes of a lead in an exemplary scenario;

FIG. 11 illustrates a recording a neural potential using a bipolar configuration, according to an implementation of the present technology;

FIG. 12 illustrates a recorded neural potential when the reference electrode is unaffected by the neural stimulus;

FIG. 13A illustrates a configuration of stimulus and sensing electrodes in the electrode assembly;

FIG. 13B illustrates an alternative configuration of stimulus and sensing electrodes in the electrode assembly;

FIG. 13C illustrates yet another alternative configuration of stimulus and sensing electrodes in the electrode assembly;

FIG. 13D illustrates a method of determining a reference electrode from the electrodes associated with the lead;

FIG. 13E illustrates a method of determining a reference electrode from the electrodes associated with the lead;

FIG. 14A illustrates a method to select a reference electrode to record a neural potential;

FIG. 14B illustrates an alternative method to select a reference electrode to record a neural potential;

FIG. 14C illustrates yet another alternative method to select a reference electrode to record a neural potential; and

FIG. 14D illustrates another method to select a reference electrode to record a neural potential.

### DETAILED DESCRIPTION OF THE PRESENT TECHNOLOGY

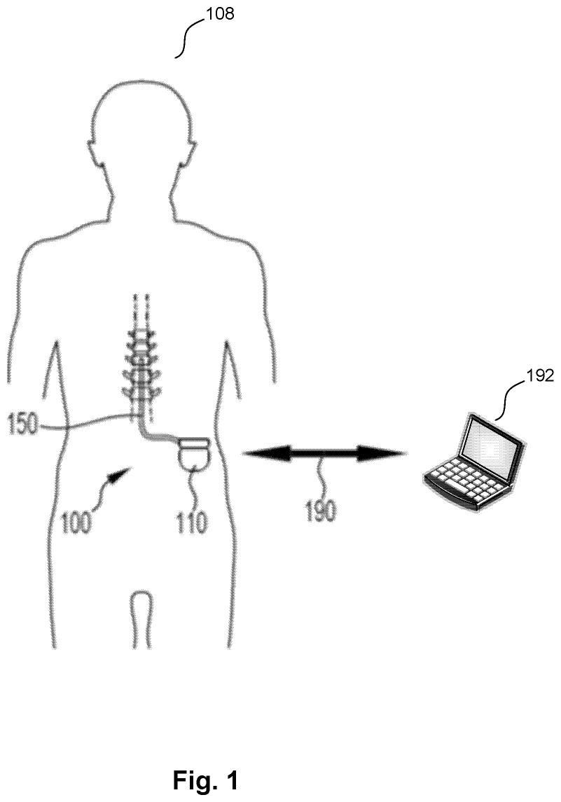

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array or assembly 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

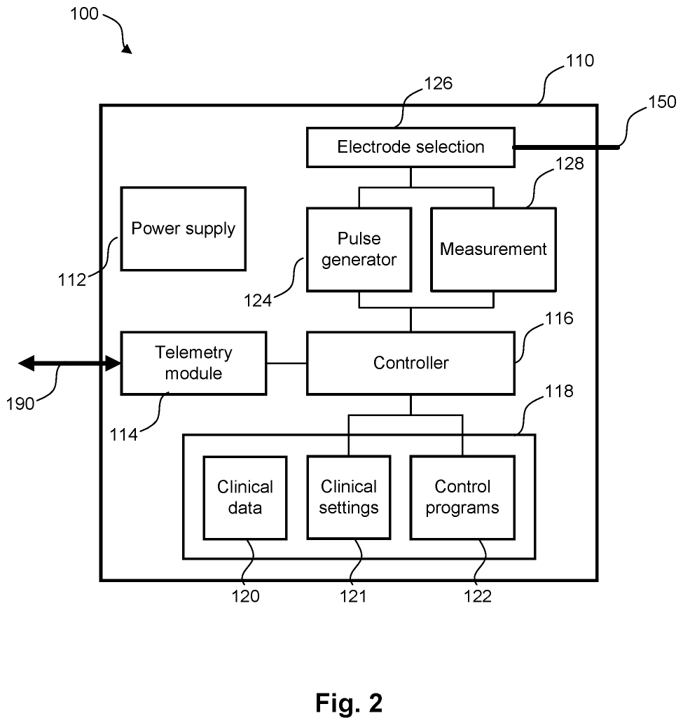

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, patient settings 121, control programs 122, and the like. Controller 116 controls a pulse generator 124 to generate stimuli, such as in the form of pulses, in accordance with the patient settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement (sensing) electrode(s) of the electrode array 150 as selected by electrode selection module 126.

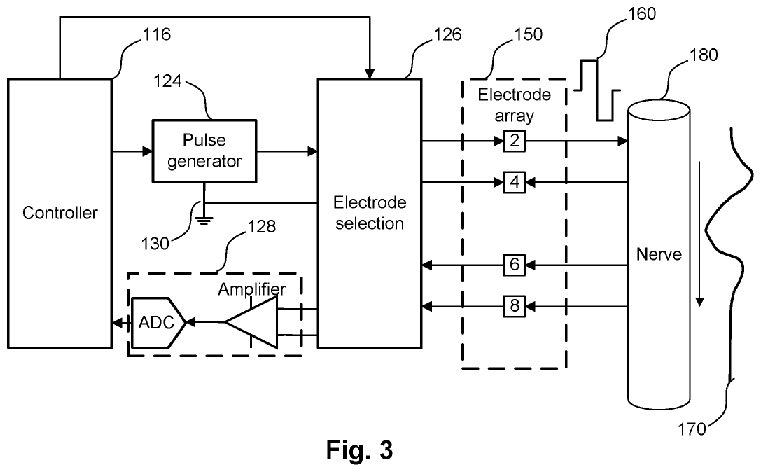

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulation electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulation electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus and/or return electrodes. The set of stimulus and return electrodes and their respective polarities is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain, the clinician nominates that configuration for ongoing use. The therapy parameters may be loaded into the memory 118 of the stimulator 100 as the clinical settings 121.

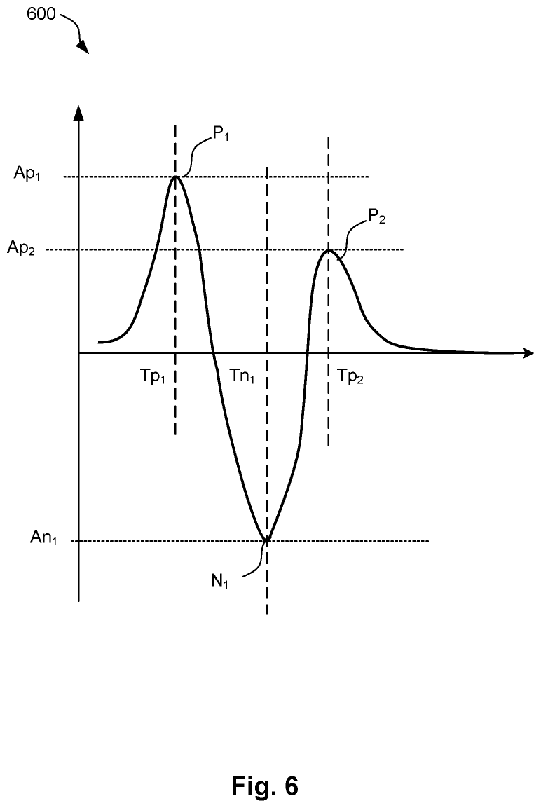

FIG. 6 illustrates the typical form of a single-ended ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The measurement circuitry 128 for example may operate in accordance with the teachings of International Patent Application Publication No. WO2012155183 by the present applicant, the content of which is incorporated herein by reference.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO 2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, stimulation settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

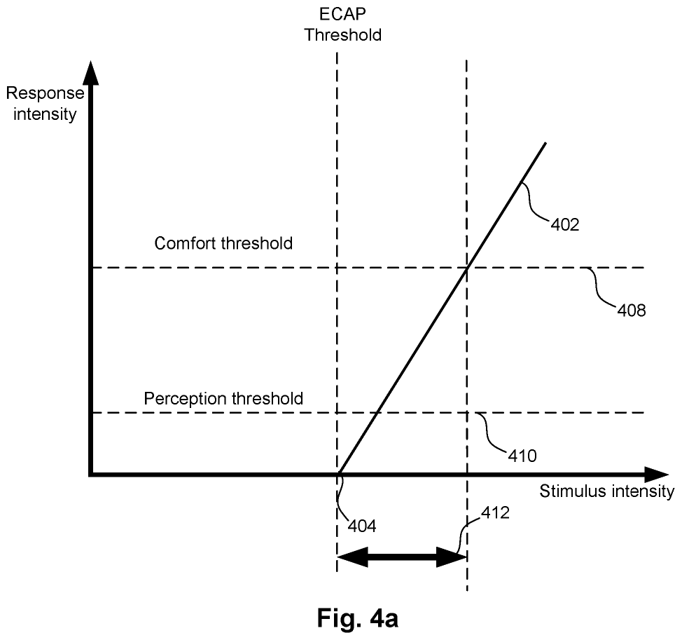

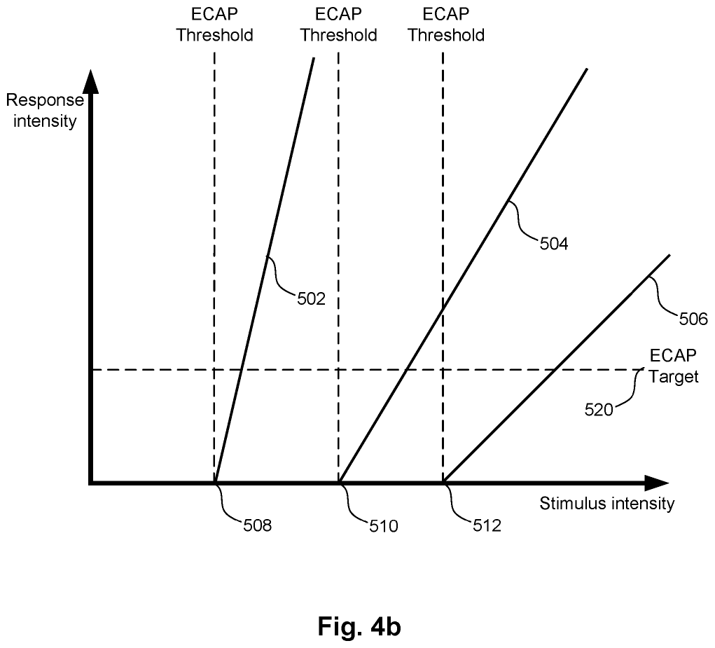

An activation plot, or growth curve, is an approximation to the relationship between stimulus intensity (e.g. an amplitude of the current pulse 160) and intensity of neural response 170 resulting from the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment: if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, Tis the ECAP threshold and S is the slope of the activation plot (referred to herein as the patient sensitivity). The slope S and the ECAP threshold T are the key parameters of the activation plot 402.

FIG. 4a also illustrates a discomfort threshold 408, which is an ECAP amplitude above which the patient 108 experiences uncomfortable or painful stimulation. FIG. 4 also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and evokes an ECAP amplitude that is below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP value and a measured ECAP amplitude and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulus (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

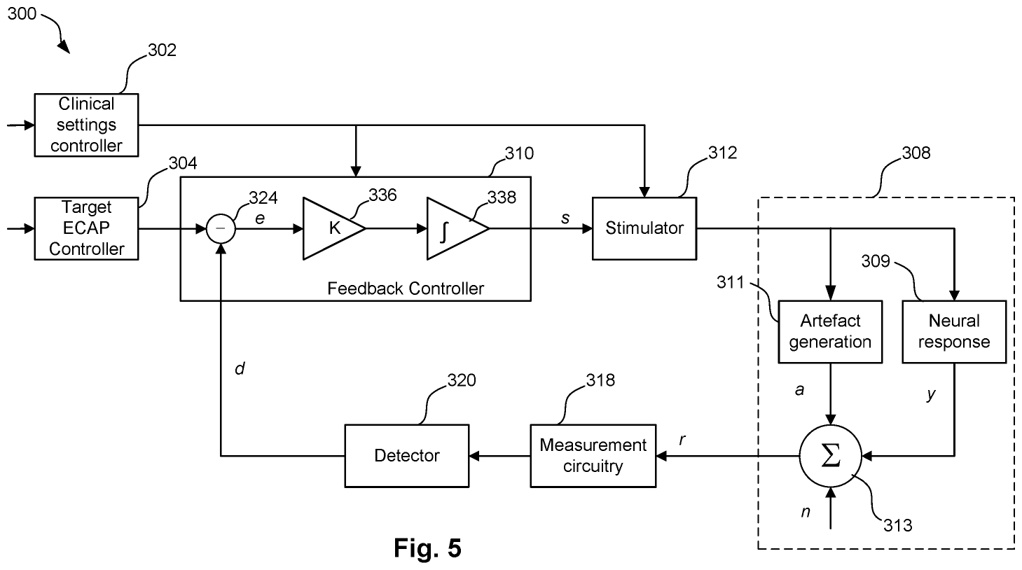

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including electrical noise from external sources such as 50 Hz mains power: electrical disturbances produced by the body such as neural responses evoked not by the device but by other causes such as peripheral sensory input, EEG, EMG, and electrical noise from amplifier 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise), and samples the amplified sensed signal r to capture a “signal window” comprising a predetermined number of samples of the amplified sensed signal r. The ECAP detector 320 processes the signal window and outputs a measured neural response intensity d. A typical number of samples in a captured signal window is 60. In one implementation, the neural response intensity comprises a peak-to-peak ECAP amplitude. The measured response intensity d is input into the feedback controller 310. The feedback controller 310 comprises a comparator 324 that compares the measured response intensity d (also referred to as the feedback variable) to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP value. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, an adjustment δs to the current stimulus intensity parameter s may be computed by the feedback controller 310 as

$$s = \int Kedt \qquad ( 2 )$$

where K is the gain of the gain element 336 (the controller gain).

A target ECAP value is input to the comparator 324 via the target ECAP controller 304. In one implementation, the target ECAP controller 304 provides an indication of a specific target ECAP value. In another implementation, the target ECAP controller 304 provides an indication to increase or to decrease the present target ECAP value. The target ECAP controller 304 may comprise an input into the neural stimulus device, via which the patient or clinician can input a target ECAP value, or indication thereof. The target ECAP controller 304 may comprise memory in which the target ECAP value is stored, and from which the target ECAP amplitude is provided to the feedback controller 310.

A clinical settings controller 302 provides clinical parameters to the system, including the gain K for the gain element 336 and the stimulation parameters for the stimulator 312. The clinical settings controller 302 may be configured to adjust the gain K of the gain element 336 to adapt the feedback loop to patient sensitivity. The clinical settings controller 302 may comprise an input into the neural stimulus device, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

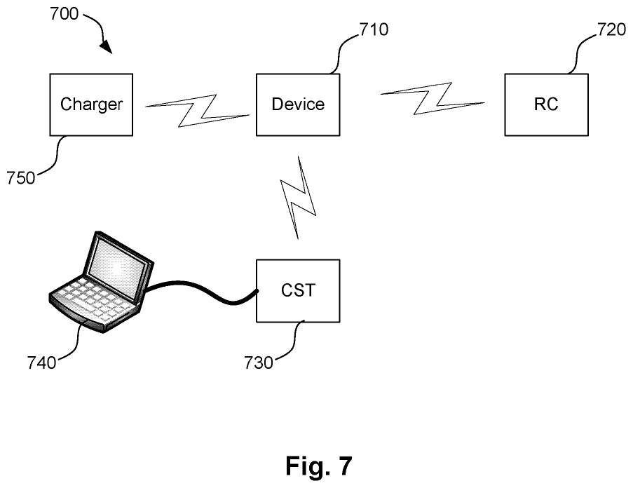

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation: adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

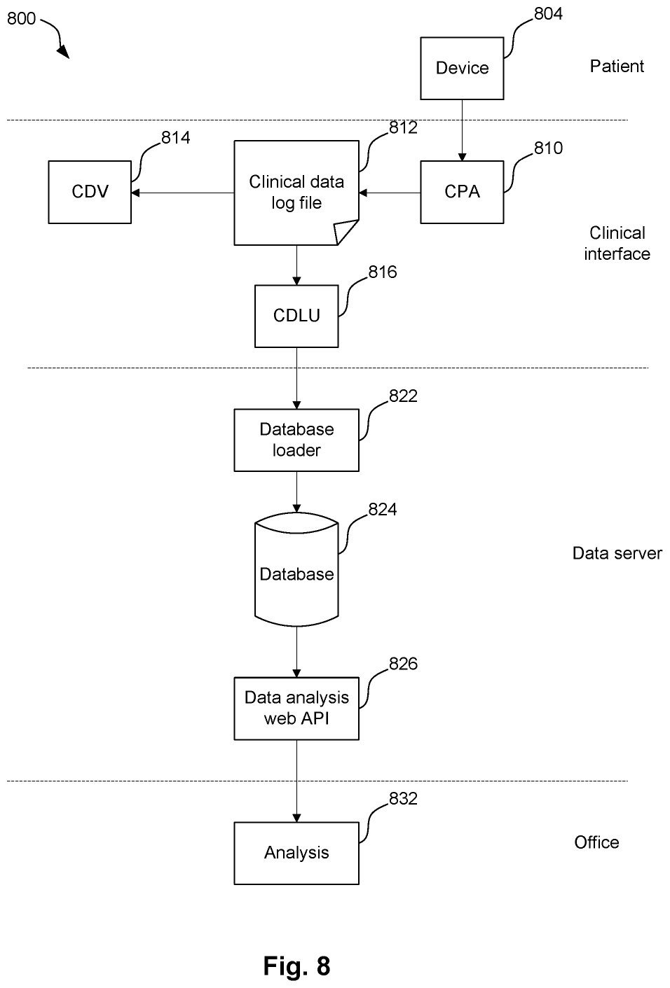

FIG. 8 is a block diagram illustrating the data flow 800 of a neuromodulation therapy system such as the system 700 of FIG. 7 according to one implementation of the present technology. Neuromodulation device 804, once implanted within a patient, applies stimuli over a potentially long period such as weeks or months and records neural responses, stimulation settings, paraesthesia target level, and other operational parameters, discussed further below. Neuromodulation device 804 may comprise a Closed-Loop Stimulator (CLS), in that the recorded neural responses are used in a feedback arrangement to control stimulation settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing the stimulation parameters for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session: (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 and/or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user or clinician with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service that runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web API 826 which provides data for third-party analysis such as by the analysis module 832, located remotely from the data server. The ability to obtain, store, download and analyse large amounts of neuromodulation data means that the present technology can: improve patient outcomes in difficult conditions: enable faster, more cost effective and more accurate troubleshooting and patient status; and enable the gathering of statistics across patient populations for later analysis, with a view to diagnosing actiologies and predicting patient outcomes.

### Lead Structure

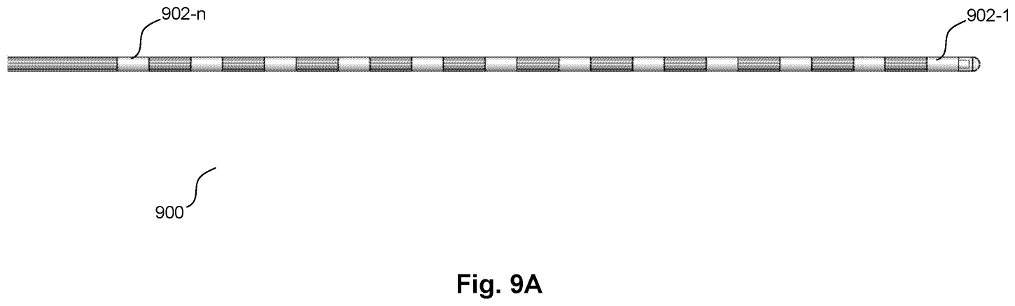

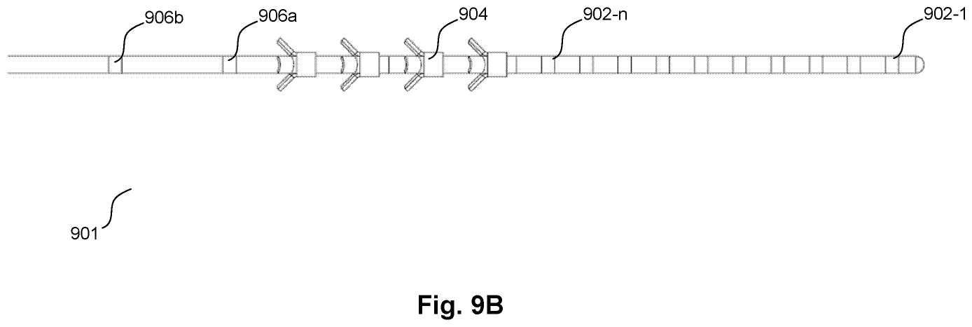

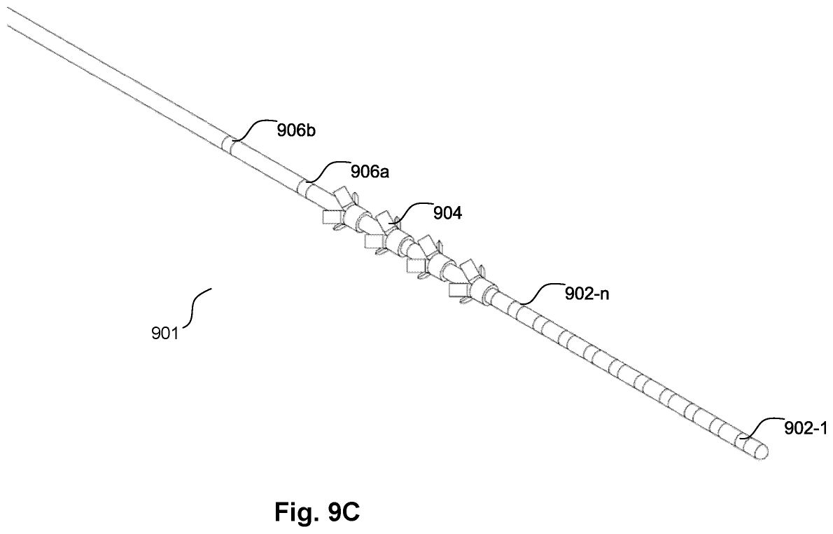

FIG. 9A is a side view of an electrode assembly (array) 900 for stimulating the dorsal column, according to one implementation of the present technology. The electrode assembly 900 includes an elongated longitudinal body having a proximal end and a distal end. The longitudinal body is fabricated from a biocompatible material such as epoxy and comprises several planetary lumens within the longitudinal body. The construction of the lead body is well known in the art. FIG. 9A illustrates electrode contacts 902-1 (most distal) to 902-n (most proximal) that can be configured in a stimulation mode and/or in a sensing mode. Further, in a stimulation mode, the electrode contacts 902-1 to 902-n can be configured as a stimulation electrode and a return electrode, whereas in a sensing mode, the electrode contacts 902-1 to 902-n may be configured as recording electrode and a reference electrode. The electrode assembly shown in FIG. 9A includes 12 electrode contacts, however, the present technology applies to electrode assemblies with a varying number of electrodes.

FIG. 9B illustrates a side view of an electrode assembly 901 for stimulating the pelvic floor nerves, according to one implementation of the present technology. The distal end of the electrode body 901 comprises a first set of electrodes 902-1 to 902-n. In the implementation shown in FIG. 9B, the first set of electrodes includes 10 contacts. Further, the electrode assembly comprises a plurality of anchoring elements 904. For instance, there are four anchoring elements 904 according to one implementation. The anchoring elements may be referred to as tines. The anchoring elements or tines 904 aid in affixing the lead to the tissue. The structure of the anchoring elements 904 are well known in the art. FIG. 9C is a perspective view of the electrode assembly 901. Furthermore, electrode assembly 901 includes a second set of electrodes 906a and 906b that are separated by distance and by the anchoring elements 904 from the first set of electrodes. A person skilled in the art may appreciate that the number of electrodes and anchoring elements may vary according to the requirements. In an exemplary implementation, the separation between the first and second sets of electrodes may be between 10 mm and 20 mm. The first set and the second set of electrodes are configurable in at least one of a stimulation mode and a sensing mode. The electrodes configured in the stimulation mode include at least one stimulation electrode and at least one return electrode, where the electrodes configured in the stimulation mode apply an electrical stimulus to a portion of tissue. Further, the electrodes configured in the sensing mode are configured to measure a neural potential. The electrodes configured in the sensing mode include at least one recording electrode and at least one reference electrode.

### Nerve to Electrode Distance

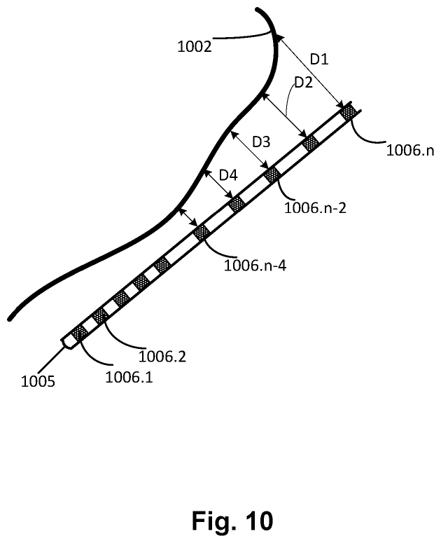

Target nerves such as the vagus nerve and the sacral nerve which are modulated in neuromodulation therapies can extend in various ways within the anatomy. The way in which the target nerves are disposed within the anatomy plays are crucial role in the selection of the reference electrode. FIG. 10 illustrates the distance between the nerve and the electrodes of an electrode array in an exemplary scenario. A target nerve 1002 extends in a random manner within a certain part of the anatomy of the patient. Further, a lead or electrode array 1005 having electrode contacts 1006.1 to 1006.n extends alongside the target nerve 1002. It can be observed in FIG. 10 that the target nerve 1002 diverts away from the lead 1005. The target nerve to electrode distance varies along the electrodes 1006.1-1006.n. Further, the target nerve to electrode distance D1 is the highest at the electrode contact 1006.n. Further, the target nerve-to-electrode distances D2, D3 and D4 are progressively shorter as compared with D1. Therefore, it can be inferred that the neural response sensed at electrode 1006.n will be the lowest in intensity, compared to other electrodes on the electrode array 1005, and is a preferred candidate to be configured as a reference electrode.

In some cases, the target nerve may be disposed in a manner that the electrode next to the stimulus electrodes has the highest and/or maximum target nerve-to-electrode distance. In such a case, the electrode close to the stimulus electrodes may be chosen as the reference electrode as the chances of detecting the elicited neural responses are less. Therefore, the reference electrode need not be the electrode most distant from the stimulation site.

The ECAP threshold at an electrode varies monotonically with the distance between the electrode and the target nerve, since the smaller the distance, the smaller is the ECAP threshold. Therefore, in one implementation, the ECAP threshold may be measured at each electrode by configuring it as a stimulation electrode and measuring the ECAP growth curve as described above. The ECAP threshold at that electrode may then be used as a proxy for the distance between the electrode and the target nerve. Other techniques for estimating the distance between a stimulation electrode and the target nerve are disclosed in International Patent Publication no. WO2016/161484 by the present applicant, the entire contents of which are herein incorporated by reference.

### Bipolar Recording Vs Monopolar Recording

FIG. 11 illustrates a configuration 1100 for recording neural potential using a bipolar recording configuration, according to an implementation of the present technology. In closed-loop neural stimulation therapy, the amplitude of the neural response is a crucial value to adjust the stimulation parameter. Therefore, a bipolar recording method may be employed to measure an accurate value of the peak-to-peak amplitude of the neural response. FIG. 11 shows an exemplary electrode array, wherein electrodes 1102, 1104 and 1106 are configured in a stimulation mode.

The electrodes 1102, 1104 and 1106 may be activated to apply a tripolar stimulus pattern to the tissue of a patient. Further, electrodes 1102 and 1106 may be configured to apply anodic pulses, whereas electrode 1104 is configured to apply a cathodic pulse. The tripolar stimulation is performed using the methods in the International Patent publication no. WO2020082118A1, the content of which is incorporated herein in its entirety by reference. In an instance, the electrodes of the electrode array may act sequentially as stimulation electrodes and return electrodes during the application of the triphasic stimulus.

Now, electrodes 1108 and 1110 are configured in the sensing mode as sensing electrodes, wherein electrode 1108 is the recording electrode and electrode 1110 is the reference electrode. Electrodes 1108 and 1110 are connected to the non-inverting and inverting ends of a differential amplifier, which is a part of the measurement circuitry 128. In an instance, electrode 1108 may record an evoked neural potential 1112 in response to the application of the stimulus. Further, electrode 1110, which is the reference electrode, records a different neural potential 1114 as it is at a further distance from the stimulus electrodes. In the case of SCS, we are interested in the amplitude of the fast response or the evoked compound action potential (ECAP): the measured signal 1116 is a difference between the recorded signal 1112 and the reference signal 1114. The reference signal 1114 is similar to the recorded signal 1112 but diminished due to the increased distance from the stimulus electrodes. The measured signal 1116, therefore, is distorted to an extent but includes an accurate amplitude value of the ECAP. In measured signal 1116, the peak-to-peak amplitude 1117 may be clearly seen.

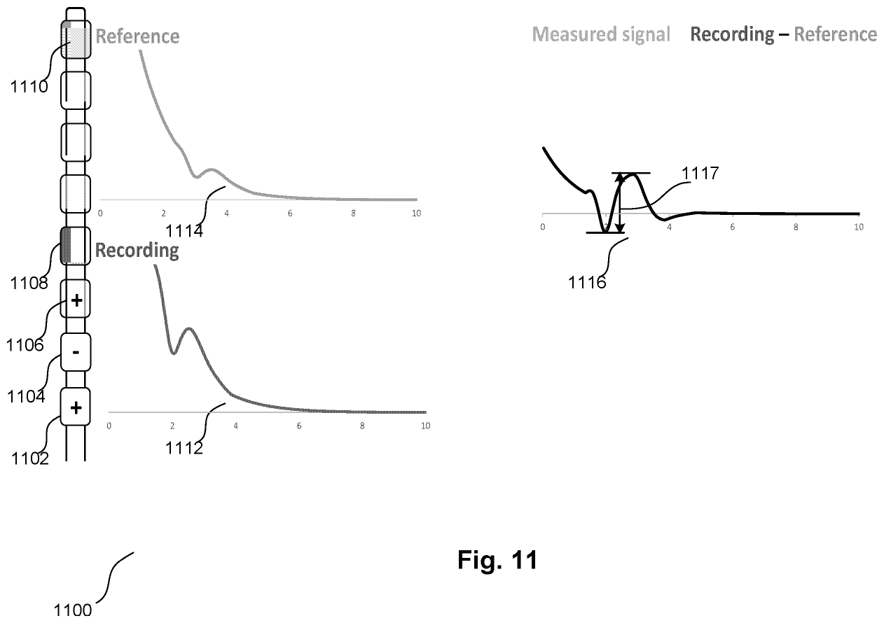

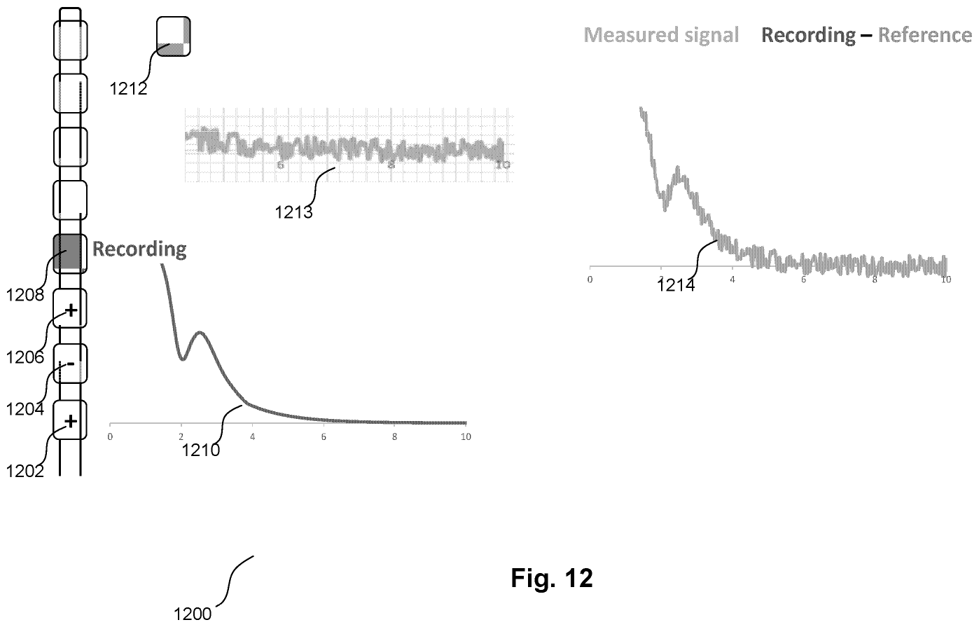

FIG. 12 illustrates a monopolar configuration 1200 for recording neural potential with the reference electrode unaffected by the neural stimulus. In FIG. 12, the electrodes 1202, 1204 and 1206 are configured as stimulus electrodes for applying neural stimulation to the tissue. The stimulus elicits a neural potential from the corresponding region of the tissue. Further, electrode 1208 is configured as a recording electrode, and electrode 1212 is configured as a reference electrode. The reference electrode 1212, in this configuration, is chosen such that the reference electrode 1212 senses an insubstantial amount of the elicited neural response. For example, the reference electrode 1212 may be chosen to be an electrode on the array that captures an insubstantial amount of the elicited neural response. The value of an insubstantial amount of neural response is 0 to a few milli-volts or less than 5% of the signal sensed at the recording electrode 1208. In another example, the reference electrode may be chosen to be a sticky pad on the patient's arm. In this case, the reference electrode 1212 records nothing but the characteristic noise 1213, which does not vary with the application of a neural stimulus. The characteristic noise 1213 is caused by the stray biopotentials within the tissue. Therefore, the reference electrode 1212, which is the skin patch electrode, is indifferent to the elicited neural response. Alternatively, the reference electrode 1212 may be a case electrode positioned sufficiently away from the stimulus site.

As a result, the differential recording 1214 may be substantially similar to the signal 1210 recorded at the recording electrode 1208. Monopolar recording therefore has the potential to capture a single-ended ECAP. The difference from bipolar recording is that the recording 1214 is noisy due to the characteristic noise 1213 recorded by the reference electrode 1212. Monopolar or single-ended recording is preferred in the case of stimulating anatomies with mixed nerves as there is a need to analyse the neural response in its entirety and not just measure the amplitude. The single-ended neural response recorded with a reference electrode that senses an insubstantial amount of evoked neural response provides details of the constituents of neural recruitment more clearly than that of a recording with a reference electrode close to the stimulus source. Analysing the recruited fibres becomes important while stimulating mixed nerve anatomy such as the pelvic floor.

### Electrode Configurations

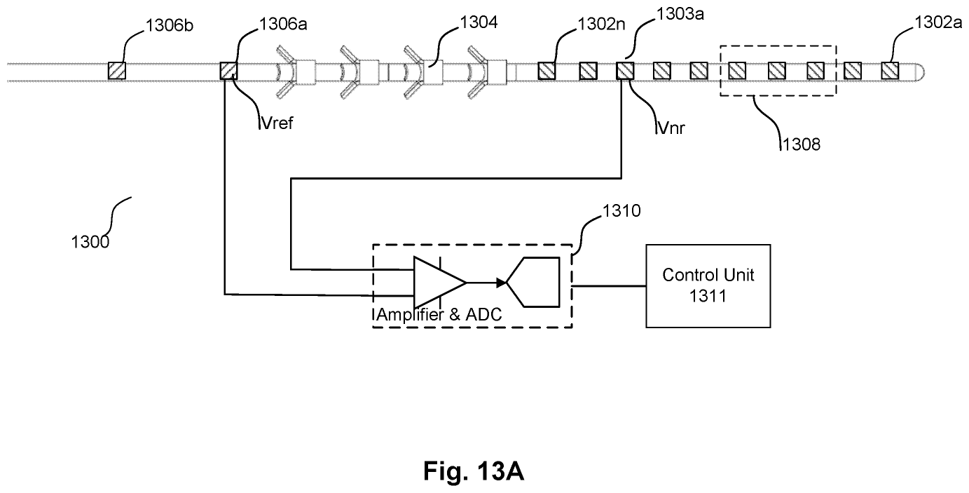

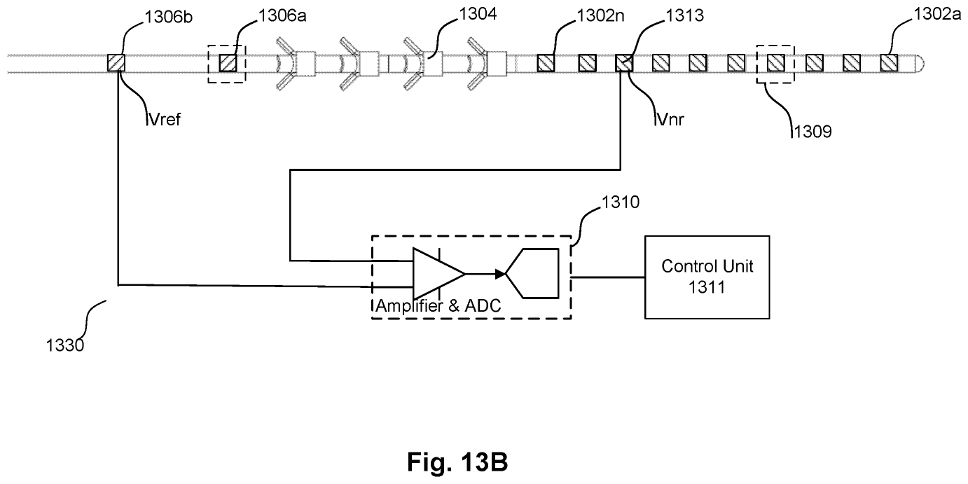

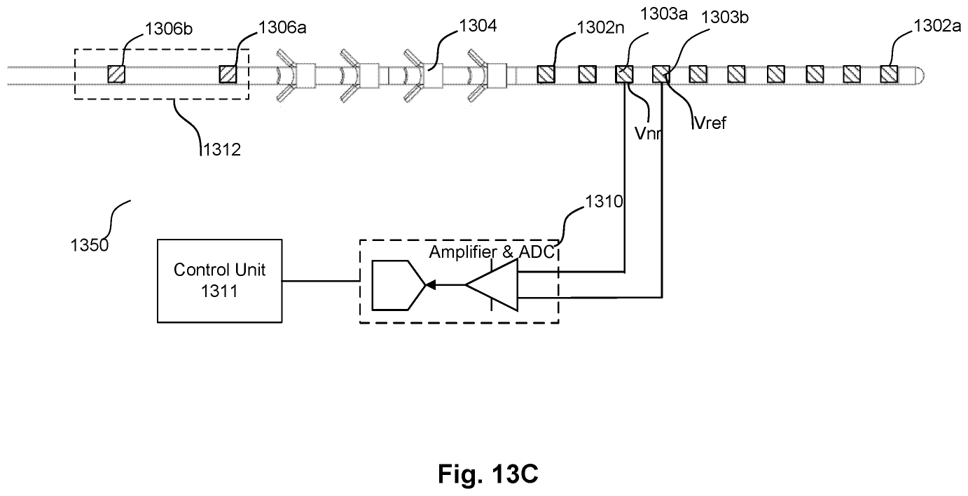

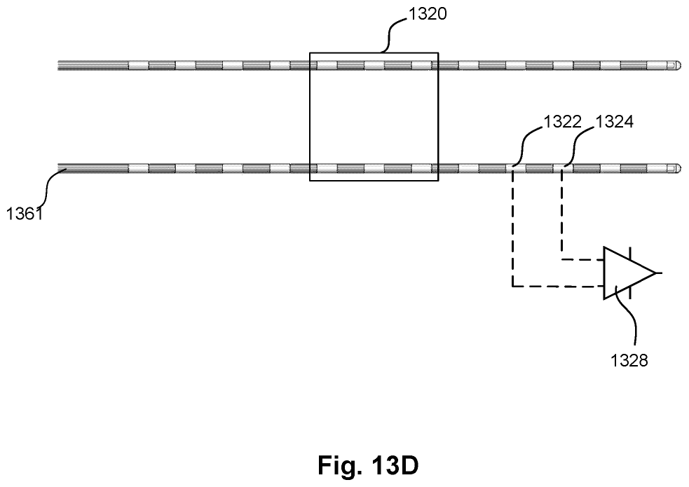

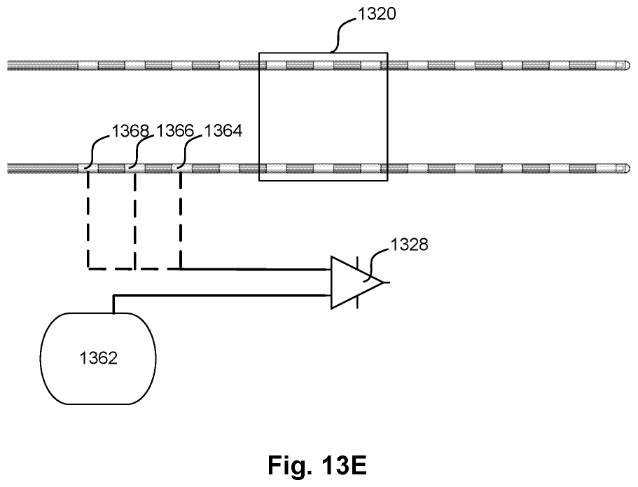

FIG. 13A illustrates a configuration 1300 of stimulus and sensing electrodes in the electrode assembly. The electrode assembly in the configuration 1300 illustrates a set of electrodes 1308 configured as stimulus electrodes, including the stimulation and return electrodes. Further, the electrode assembly 1300 includes one or more anchoring elements 1304. Further, one electrode 1305 from the first set of electrodes 1302a to 1302n is selected as a recording electrode, while the electrode 1306a from a second set of electrodes 1306a and 1306b is selected as the corresponding reference electrode. Further, the recording electrode 1305 senses a voltage of the neural response (Vnr) and the reference electrode 1306a senses a voltage Vref. Finally, the recording electrode 1305 and the reference electrode 1306a are connected to the inverting and the non-inverting terminals, or vice versa, of a differential amplifier. The measurement circuitry 1310 includes a differential amplifier and an Analog-to-digital converter (ADC).

The reference electrode 1306a is positioned distant from the stimulus electrodes 1308, such that the reference electrode 1306a captures an insubstantial amount of the elicited neural response. In sacral nerve stimulation, the reference electrode 1306a, after the anchoring elements 1304, is located within the patient's adipose tissue and/or the fascia. For this reason, the reference electrode 1306a may be further isolated from the electrical perturbations caused by the stimulus electrodes 1308. The position of the electrodes 1306a and 1306b in the adipose tissue and/or the fascia may be another factor in choosing these electrodes as reference electrodes. The reference electrode 1306a records a characteristic noise 1213 like the electrode 1212 in FIG. 12. This arrangement may be preferred in some cases where there is a need to analyse the composition of the recruited fibres.

FIG. 13B illustrates an alternative configuration 1330 of stimulus and sensing electrodes in the electrode assembly. The electrode configuration 1330 in FIG. 13B is for applying monopolar stimulation to the tissue. As illustrated, electrode 1309 is configured as the stimulation electrode, and electrode 1306a is configured as the return electrode. Further, electrode 1313 is configured as the recording electrode that senses the voltage of the neural response Vnr. Furthermore, electrode 1306b is configured as the reference electrode for providing a reference to the recording of the neural response. In some implementations, the can or a skin patch electrode may be configured as the return electrode instead of electrode 1306a.

In an example, the reference electrode is selected based on the intensity of the neural stimulation applied to the tissue. For example, suppose the intensity of the stimulus pulses are high. In that case, the reference electrode may be selected further from the stimulation electrode. Conversely, the reference electrode may be closer to the stimulation electrode if stimulus intensity is low.

In configuration 1330, the monopolar stimulation causes the stimulus field to be spread out due to the location of the return electrode 1306a away from the stimulation electrode 1309. An advantage of the field being spread out is that one can clearly distinguish the components of neural recruitment. Further, the neural response recording configurations 1300 shown in in FIG. 13A and 1330 shown in FIG. 13B is also referred to as ‘single-ended’ recording. In the single-ended recording, the reference electrode does not sense the neural response, and the recorded neural response is predominantly dependent on the recording electrode which is close to the stimulation electrode. Single-ended recordings do not distort the neural response as a double-ended recording, where both the recording electrode and the reference electrode are closer to the stimulus electrodes, may do.

Further, measurement circuitry 1310 shares the neural recording to the control unit 1311 for further processing. For example, the control unit 1311 may apply signal processing algorithms to eliminate the noise in the neural recording. Further, the control unit 1311 may be configured to analyse the morphological and spectral components of the neural recording and take appropriate actions. In an instance, the control unit may adjust the stimulus parameters based on the characteristics of the neural recording, such as, but not limited to, conduction velocity, latency, dilation, peak-to-peak ratio and amplitude. In some instances, frequency domain characteristics of the neural response may be used to configure the reference electrode. In another instance, the control unit 1311 may change a stimulus location based on the fibres identified in the neural response.

FIG. 13C illustrates another alternative configuration 1350 of stimulus and sensing electrodes in the electrode assembly. In this configuration, electrodes from the second set of electrodes, 1306a and 1306b, are configured as the stimulus electrodes 1312. The electrode contacts 1303a and 1303b from the first set of electrodes 1302a to 1302n are configured as the recording and reference electrodes respectively. It is to be noted that the electrodes of the electrode assembly 900 can be configured as the stimulus or recording electrodes. The electrode array 900 provides the flexibility to stimulate tissue and sense the neural response along the length of the electrode. Further, this is an advantage in the case of lead migration, as the changes in the electrode position relative to the anatomy may necessitate changing the stimulus and sensing positions.

FIG. 13D illustrates a method of determining a reference electrode from the electrodes associated with the electrode array 1361. In this method, a pair of electrodes other than the stimulus electrodes 1320 are connected to the measurement circuitry to sense the output. The electrode pair is swept through the entire electrode array and the results are stored. One of the electrodes from the electrode pair that results in a single-ended ECAP is chosen as a reference electrode. For example, the electrodes 1322 and 1324, which are close to the stimulus electrodes 1320, are connected to the measurement amplifier 1328. The output of the measurement amplifier 1328 is measured and recorded. In case the target nerve is deviating from the electrode array 1361 near the electrodes 1322 and 1324, the output of the measurement amplifier 1328 will be a certain non-zero number which may indicate a noise level. In this case, the electrodes 1322 and 1324 do not sense a neural response as the target nerve deviates away from the electrodes 1322 and 1324. Therefore, one of the electrodes 1322 or 1324 may be selected as a reference electrode though it may be close to the stimulus electrodes 1320.

FIG. 13E illustrates yet another method of determining a reference electrode from the electrodes associated with the electrode array. In FIG. 13E, a case electrode 1362 is selected as a reference electrode to determine the electrode that is suitable to be assigned as a reference electrode during measurement of the neural response. As the case electrode 1362 is completely indifferent to the environment in which the stimulation is occurring, it serves as an ideal electrode for evaluating a reference electrode. In some implementations, the case electrode 1362 may be chosen as the reference electrode during measurement. However, using the illustrated method, a reference electrode is being chosen from the electrode array. The recording electrode is swept through the entire electrode array (excluding the stimulus electrodes 1320) and the neural response output of the measurement amplifier 1328 is measured and recorded. As illustrated in FIG. 13E, electrodes 1364, 1366, and 1368 are used successively as recording electrodes. The electrode from the electrode array with the least neural response output from the measurement amplifier 1328 is chosen as the reference electrode, as such a reference electrode is best suited to providing a single-ended ECAP regardless of which electrode is selected as the recording electrode.

### Remote Device for Selecting the Reference Electrode

In some implementations, a remote device, such as a remote control 720 of FIG. 7, or a clinical interface 740 such as a smartphone, in communication with an implantable device may be used to select the reference electrode. The remote device may include a processing unit configured to process instructions and a communication unit configured to communicate with the implantable device. The communication unit is configured to send and receive instructions to and from the implantable device.

The remote device, for instance, may be configured to configure a plurality of electrodes of the first and second set of electrodes in at least one of a stimulation mode and a sensing mode, where the electrodes configured in the stimulation mode comprise a stimulation electrode and a return electrode, where the sensing electrodes comprise a recording electrode and a reference electrode. Further, the user may use the remote device to select the reference electrode based on the location of at least one of the stimulation electrode and the return electrode.

Referring back to FIG. 7, remote devices such as clinical interface 740, remote control 720, in communication with an implantable device, include a processing unit configured to receive instructions from a user. The remote devices further include a communication unit configured to send and receive instructions to the implantable device and can receive and send instructions to the implantable device. Further, the remote device may send instructions to configure a plurality of electrodes of the first and second set of electrodes in at least one of a stimulation mode and a sensing mode. Further, the electrodes configured in the stimulation mode comprise a stimulation electrode and a return electrode, wherein the sensing electrodes comprise a recording electrode and a reference electrode. Further, the remote device communicates instructions to the implantable device to automatically select the reference electrode based on the location of at least one of the stimulation electrodes and the return electrode.

### Method Steps for Selecting the Reference Electrode

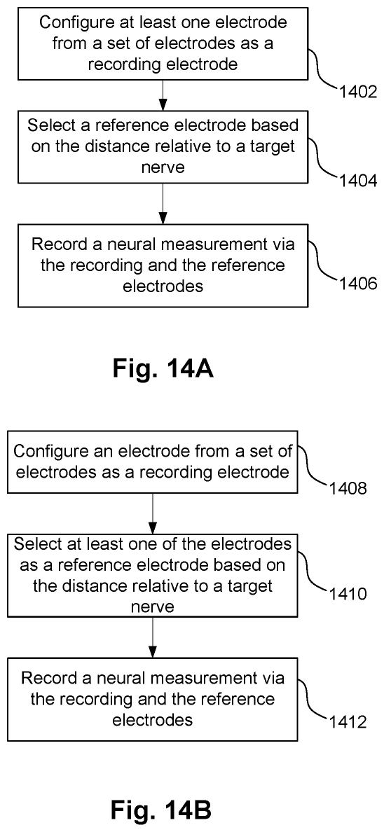

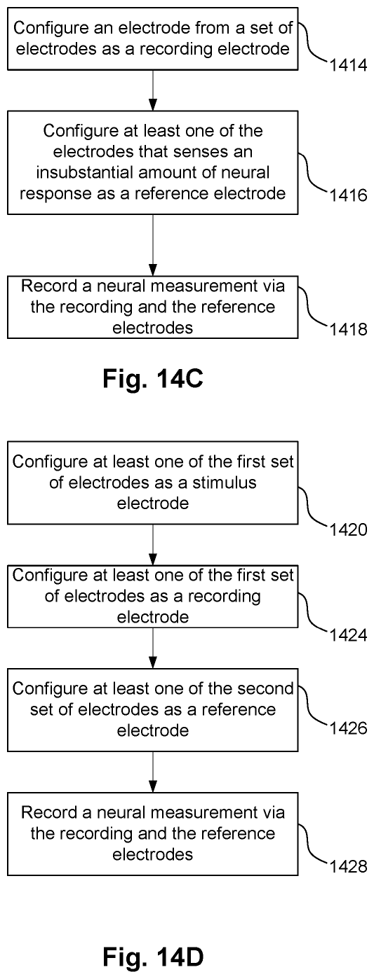

FIG. 14A illustrates a method of selecting a reference electrode to record a neural potential. At step 1402, an electrode from a set of electrodes is configured in a sensing mode as a recording electrode. At step 1404, an electrode from the set of electrodes, other than the recording electrode, is selected and configured in a sensing mode as a reference electrode based on the distance of the electrode from a target nerve. For example, the target nerve may be an individual nerve or a group of nerves. In one specific example, the target nerve may be a sacral nerve. At step 1406, a neural measurement is recorded via the recording and the reference electrodes. For performing monopolar recording, the reference electrode is selected such that the reference electrode is the farthest from the target nerve. In some implementations, the location of the reference electrode may be chosen based on the characteristics of the neural response recorded at the location. If the physician can discern the components of the neural response, which means the recordings are of a good quality, then the configuration is finalised. Alternatively, if the neural response is not of acceptable quality, then the location of the reference electrode may be changed until the neural response is of acceptable quality.

FIG. 14B illustrates an alternative method to select a reference electrode to record a neural potential. At step 1408, an electrode from a set of electrodes is configured in a sensing mode as a recording electrode. At step 1410, an electrode from the set of electrodes, other than the recording electrode, is selected and configured in a sensing mode as a reference electrode based on the distance of the electrode relative to the target nerve. The target nerve could be a nerve such as a vagus nerve or a pelvic nerve. At step 1412, a neural measurement is recorded via the recording and the reference electrodes. The electrodes in the set of electrodes on a lead could be at varying distances from the target nerve based on the way the nerve is innervated in the anatomy of the patient. For example, in sacral nerve stimulation, the lead at one point enters the region in the anatomy that is away from the sacral nerve. The lead array is surrounded by adipose tissue, bones and/or muscles. The electrodes in this portion of the array are essentially indifferent to the activity at the stimulation site. Therefore, the electrodes that are situated away from the target nerve and in an environment insulated from the stimulation activity are preferred candidates to be configured as reference electrodes.

FIG. 14C illustrates yet another alternative method to select a reference electrode to record a neural potential. At step 1414, an electrode from a set of electrodes is configured in a sensing mode as a recording electrode. At step 1416, an electrode from the set of electrodes, other than the recording electrode, that senses an insubstantial amount of neural response is selected and configured in a sensing mode as a reference electrode. An electrode may sense an insubstantial amount of neural response if, for example, the target nerve is not substantially parallel to the electrode. In another case, an electrode may sense an insubstantial amount of neural response if the nerve veers away from the electrode due to the innervation in the anatomy. This situation represents an opportunity to select a preferred reference electrode. At step 1418, a neural measurement is recorded via the recording and the reference electrodes.

FIG. 14D illustrates another method to select a reference electrode to record the neural potential. At step 1420, at least one of the first set of electrodes is configured in a stimulation mode as a stimulus electrode. At step 1424, an electrode from the first set of electrodes is selected and configured in a sensing mode as a recording electrode. For example, the recording electrode may be any electrode from the first set of electrodes based on the location of the stimulus electrodes. At step 1426, at least one of the second set of electrodes is selected and configured in a sensing mode as a reference electrode. The reference electrode senses an insubstantial amount of the elicited neural response. The reference electrode is unaffected by the neural response. In another example, an electrode from the first or the second set of electrodes is selected as a reference electrode based on the distance of an electrode from a target nerve. The distance of an electrode from the target nerve is computed using the techniques mentioned above. At step 1428, a neural measurement is recorded via the recording electrode and the reference electrode. Such a recording is not distorted, and a user may determine the recruited neural components of the neural potential.

The method steps illustrated in FIGS. 14A-14D may be implemented by the electrode selection module 126 and/or the controller 116 disclosed in FIG. 2.

It will be appreciated by persons skilled in the art that numerous variations and/or modifications may be made to the invention as shown in the specific implementations without departing from the spirit or scope of the invention as broadly described. The present implementations are, therefore, to be considered in all respects as illustrative and not limiting or restrictive.

### LABEL LIST

stimulus electrodes 2
return electrode 4
measurement electrodes 6
measurement reference electrode 8
spinal cord stimulator 100
patient 108
electronics module 110
battery 112
telemetry module 114
controller 116
memory 118
clinical data 120
patient settings 121
control programs 122
pulse generator 124
electrode selection module 126
measurement circuitry 128
system ground 130
electrode array 150
biphasic stimulus pulse 160
ECAPs 170
nerve 180
transcutaneous communications channel 190
external computing device 192
CLNS system 300
clinical settings controller 302
target ECAP controller 304
box 308
box 309
feedback controller 310
box 311
stimulator 312
element 313
signal amplifier 318
ECAP detector 320
comparator 324
gain element 336
integrator 338
activation plot 402
ECAP threshold 404
comfort threshold 408
perception threshold 410
therapeutic range 412
activation plots 502
respective activation plots 506
ECAP thresholds 508
ECAP thresholds 510
ECAP thresholds 512
ECAP target 520
ECAP 600
neuromodulation system 700
neuromodulation device 710
remote controller 720
clinical System Transceiver 730
clinical Interface 740
charger 750
data flow 800
neuromodulation device 804
clinical programming application 810
clinical data log file 812
Clinical data viewer 814
clinical Data Uploader 816
database loader 822
database 824
data analysis web API 826
analysis module 832
electrode assembly 900
electrode assembly 901
electrode 902-1
electrode 902-n
anchoring units 904
electrode 906a
electrode 906b
target nerve 1002
electrode array 1005
electrode 1006.1
electrode 1006.n
configuration 1100
electrodes 1102
electrode 1104
electrode 1106
electrode 1108
electrode 1110
neural potential 1112
reference signal 1114
measured signal 1116
amplitude 1117
configuration 1200
electrode 1202
electrode 1204
electrode 1206
recording electrode 1208
reference electrode 1212
characteristic noise 1213
recording 1214
electrode assembly 1300
electrode 1302a
electrode 1302n
recording electrode 1303a
reference electrode 1303b
anchoring units 1304
recording electrode 1305
electrode 1306a
electrode 1306b
stimulus electrodes 1308
stimulus electrode 1309
measurement circuitry 1310
control unit 1311
stimulus electrodes 1312
recording electrode 1313
stimulus electrodes 1320
electrode 1322
electrode 1324
measurement amplifier 1328
alternative configuration 1330
alternative configuration 1350
electrode array 1361
case electrode 1362
electrode 1364
electrode 1366
electrode 1368
step 1402
step 1404
step 1406
step 1408
step 1410
step 1412
step 1414
step 1416
step 1418
step 1420
step 1424
step 1426
step 1428

1-20. (canceled)

21. A neural stimulation system comprising:
an implantable neuromodulation device for controllably delivering neural stimuli,
an electrode assembly electrically coupled to the implantable neuromodulation device, the electrode assembly including a set of electrodes proximal to a distal end of the electrode assembly,
the implantable neuromodulation device comprising:
stimulation circuitry for applying the neural stimuli to at least one target nerve, wherein the neural stimuli elicit a neural potential from a target nerve;
measurement circuitry configured to process signals sensed subsequent to respective neural stimuli at a pair of sensing electrodes of the set of electrodes; and
a control unit configured to control the stimulation circuitry to apply the neural stimuli; and
a processor configured to:
configure a plurality of electrodes of the set of electrodes in at least one of a simulation mode and a sensing mode, wherein the electrodes configured in the stimulation mode are connected to the stimulation circuitry and the electrodes configured in the sensing mode are connected to the measurement circuitry, wherein the plurality of electrodes configured in the sensing mode comprise a reference electrode; and
select an electrode from the set of electrodes as the reference electrode such that the reference electrode senses an insubstantial amount of the elicited neural potential.

22. The neural stimulation system of claim 21, wherein the plurality of electrodes configured in the sensing mode comprise a recording electrode, and wherein the reference electrode senses an insubstantial amount of the elicited neural potential if the reference electrode senses less than 5% of the magnitude of the elicited neural potential sensed at the recording electrode.

23. The neural stimulation system of claim 21, wherein the plurality of electrodes configured in the stimulation mode comprise a return electrode, and wherein the processor is further configured to select the return electrode based on a desired level of a field at the target nerve.

24. The neural stimulation system of claim 21, wherein the target nerve includes nerve fibres such as sacral nerve, vagus nerve, and nerve fibres in a dorsal column.

25. The neural stimulation system of claim 21, further comprising a remote device in communication with the implantable neuromodulation device.

26. The neural stimulation system of claim 25, wherein the processor is part of the remote device.

27. The neural stimulation system of claim 21, wherein the processor forms part of the implantable neuromodulation device.

28. A remote device in communication with an implantable neuromodulation device, the remote device comprising:
a processing unit configured to receive instructions from a user;
a communication unit configured to send and receive instructions to and from the implantable neuromodulation device,
the processing unit configured to send instructions to the implantable neuromodulation device to:
configure a plurality of electrodes of a set of electrodes in at least one of a stimulation mode and a sensing mode, wherein the electrodes configured in the sensing mode comprise a reference electrode; and
select an electrode from the set of electrodes as the reference electrode to sense an insubstantial amount of the elicited neural potential.

29. The remote device of claim 28, wherein the plurality of electrodes configured in the sensing mode comprise a recording electrode, and wherein the reference electrode senses an insubstantial amount of the elicited neural potential if the reference electrode senses less than 5% of the magnitude of the elicited neural potential sensed at the recording electrode.

30. The remote device of claim 28, wherein the electrodes configured in the stimulation mode comprise a return electrode, and wherein the processing unit is further configured to select the return electrode based on a desired level of a field at a target nerve.

31. The remote device of claim 30, wherein the target nerve includes nerve fibres such as sacral nerve, vagus nerve, and nerve fibres in a dorsal column.

32. The remote device of claim 28, wherein the remote device is one of a remote control, a portable computing device, and an external device.

33. A method of selecting a reference electrode, the method comprising:
providing stimulation circuitry and measurement circuitry;
providing a processing unit configured to control the stimulation circuitry and the measurement circuitry;
providing for a lead body having a proximal end and a distal end, the lead body having a set of electrodes proximal to the distal end;
wherein the set of electrodes are configurable in at least one of a stimulation mode and a sensing mode;
wherein the electrodes configured in the stimulation mode are connected to the stimulation circuitry, wherein the electrodes configured in the stimulation mode apply an electrical stimulus to a target nerve, wherein the electrical stimulus elicits a neural potential; and
wherein the electrodes configured in the sensing mode are connected to the measurement circuitry configured to measure the elicited neural potential, wherein the electrodes configured in the sensing mode include at least one reference electrode; and
selecting an electrode from the set of electrodes as the reference electrode such that the reference electrode senses an insubstantial amount of the elicited neural potential.

34. The method of claim 33, wherein the electrodes configured in the sensing mode comprise a recording electrode, and wherein the reference electrode senses an insubstantial amount of the elicited neural potential if the reference electrode senses less than 5% of the magnitude of the elicited neural potential sensed at the recording electrode.

35. The method of claim 33, wherein the electrodes configured in the stimulation mode include a return electrode, further comprising selecting the return electrode based on a desired level of a field at the target nerve.

36. The method of claim 33, wherein the target nerve includes nerve fibres such as sacral nerve, vagus nerve, and nerve fibres in a dorsal column.

37-40. (canceled)

41. A neural stimulation lead for applying stimulation to a tissue, the neural stimulation lead comprising:
a lead body having a proximal end and a distal end;
the lead body having a first set of electrodes at the distal end, a plurality of anchoring elements proximal to first set of electrodes and a second set of electrodes proximal to the anchoring elements;
wherein the first set of electrodes and the second set of electrodes are configurable in at least one of a stimulation mode and a sensing mode;
wherein the electrodes configured in the stimulation mode are connected to stimulation circuitry, wherein the electrodes configured in the stimulation mode apply an electrical stimulus to a target nerve, wherein the electrical stimulus elicits a neural potential;
wherein the electrodes configured in the sensing mode are connected to measurement circuitry configured to measure the elicited neural potential, wherein the electrodes configured in the sensing mode include at least one reference electrode,
wherein an electrode from the second set of electrodes is configured as the reference electrode.

42. The neural stimulation lead of claim 41, wherein the electrodes configured in the sensing mode comprise a recording electrode, and wherein an electrode from the first set of electrodes is configured as the recording electrode.

43. The neural stimulation lead of claim 41, wherein the electrodes configured in the stimulation mode include a return electrode, and wherein an electrode from the second set of electrodes is configured as the return electrode.
